(19) United States
(12) Patent Application Publication
(10) Pub. No.: US 2009/0124875 A1
(43) Pub. Date: May 14, 2009

(54) Implantable Creatinine Sensor and Related Methods

(75) Inventors: James Gregory Bentsen, North St. Paul, MN (US); Misty L. Noble, Bothell, WA (US)

Correspondence Address: PAULY, DEVRIES SMITH & DEFFNER, L.L.C., PLAZA VII- SUITE 3000, 45 SOUTH SEVENTH STREET, MINNEAPOLIS, MN, 55402-1630, US

(73) Assignee: CARDIAC PACEMAKERS, INC., St. Paul, MN (US)

(21) Appl. No.: 12/269,510

(22) Filed: Nov. 12, 2008

Related U.S. Application Data
(60) Provisional application No. 60/987,942, filed on Nov. 14, 2007.

(51) Int. Cl. A61B 5/1459

(52) U.S. Cl. 600/341; 600/322; 607/4

(57) ABSTRACT

Embodiments of the invention are related to implantable creatinine sensors and related methods, amongst other things. In an embodiment, the invention includes an implantable creatinine sensor including a sensing element. The sensing element can include a creatinine deiminase enzyme covalently bound to a substrate and a pH-indicating compound in ionic communication with the creatinine deiminase enzyme. The implantable creatinine sensor can also include an optical excitation assembly configured to illuminate the sensing element and an optical detection assembly configured to receive light from the sensing element. Other embodiments are also included herein.

This application claims the benefit of U.S. Provisional Application No. 60/987,942, filed Nov. 14, 2007, the content of which is herein incorporated by reference in its entirety.

### TECHNICAL FIELD

This disclosure relates generally to implantable sensors and, more particularly, to implantable sensors for detecting creatinine, amongst other things.

### BACKGROUND OF THE INVENTION

Creatinine is a normal breakdown product of muscle metabolism and is excreted from the body through the kidneys. It is considered to be a clinical marker of renal function. Normal serum creatinine concentrations are between 0.6 and 1.3 mg/dL of blood. When renal function declines, less creatinine is excreted from the body and serum concentrations of creatinine rise. A serum creatinine concentration higher than 3.0 mg/dL is generally believed to be an indicator of renal system failure.

Creatinine is an important clinical analyte for various heart conditions because of the dependence of proper renal function on adequate cardiac output. Specifically, creatinine is an important clinical analyte for monitoring heart failure patients. Heart failure refers to a clinical syndrome in which an abnormality of cardiac function causes a below normal cardiac output that can fall below a level adequate to meet the metabolic demand of peripheral tissues. Reduced cardiac output has a depressing effect on renal function due to decreased renal perfusion, which causes a reduction in salt and water excretion by the pressure natriuresis mechanism and results in increased fluid retention. Chronic reduced cardiac output can also lead to renal failure and a resulting increase in serum creatinine concentrations. Creatinine is also an important clinical analyte for monitoring heart failure patients because the pharmacological therapy prescribed in heart failure can accentuate electrolyte imbalance and renal insufficiency.

Typically, clinicians caring for heart failure patients evaluate serum creatinine concentration by drawing blood and then using in vitro assays. Such tests can accurately determine the serum creatinine concentration of the patient at the time of the blood draw. Unfortunately, the use of in vitro assays generally requires that the patient visit a care facility to have their blood drawn. As such, when using in vitro assays, there are practical limits to the frequency with which serum creatinine concentrations can be assessed. In addition, co-morbidities commonly associated with renal disease and/or heart disease, such as diabetes, can make frequent blood draws risky in some patient populations.

Significant efforts have been focused on developing implantable sensors. For example, attempts have been made to develop implantable glucose sensors because of the need to frequently monitor glucose levels in the serum of diabetics. However, many implantable glucose sensors have suffered from various problems including substantial signal drift over time making them generally not suitable for chronic use in the in vivo environment. It has been postulated that the formation of a fibrous capsule around the implanted sensor as a result of wound healing phenomena and the host response to the implant contributes to the observed signal drift.

### SUMMARY OF THE INVENTION

Embodiments of the invention are related to implantable creatinine sensors and related methods, amongst other things. In an embodiment, the invention includes a chronically implantable creatinine sensor. The sensor can include a sensing element comprising a creatinine deiminase enzyme covalently bound to a substrate and a pH-indicating compound in ionic communication with the creatinine deiminase enzyme. The sensing element can be configured to change optical properties in response to changes in creatinine concentrations in vivo. The sensor can include an optical excitation assembly configured to illuminate the sensing element and an optical detection assembly configured to receive light from the sensing element.

In an embodiment, the invention includes an implantable medical device. The medical device can include a pulse generator and a chemical sensor in communication with the pulse generator. The chemical sensor can be configured to detect creatinine concentration in a bodily fluid. The chemical sensor can include

a sensing element comprising creatinine deiminase covalently bound to a substrate and a pH-indicating compound in ionic communication with the creatinine deiminase.

In an embodiment, the invention can include a medical system. The medical system can include an external monitoring device and a chemical sensor in communication with the external monitoring device. The chemical sensor can be configured to detect creatinine concentration in a bodily fluid. The chemical sensor can include a sensing element comprising creatinine deiminase covalently bound to a substrate and a pH-indicating compound in ionic communication with the creatinine deiminase.

In an embodiment, the invention can include an implantable creatinine sensor. The sensor can include a sensing element comprising an enzyme with creatinine deiminase activity covalently bonded to a substrate and a pH-indicating compound in ionic communication with the creatinine deiminase. The sensor can also include an optical excitation assembly configured to illuminate the sensing element and an optical detection assembly configured to receive light from the sensing element.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope of the present invention is defined by the appended claims and their legal equivalents.

### BRIEF DESCRIPTION OF THE DRAWINGS

The invention may be more completely understood in connection with the following drawings, in which:

FIG. 1 is a schematic cross-sectional view of an implantable creatinine sensor in accordance with various embodiments of the invention.

FIG. 2 is a schematic cross-sectional view of an implantable creatinine sensor in accordance with various embodiments of the invention.

FIG. 3 is a schematic cross-sectional view of an implantable creatinine sensor in accordance with various embodiments of the invention.

FIG. 4 is a schematic cross-sectional view of an implantable creatinine sensor in accordance with various embodiments of the invention.

FIG. 5 is a schematic cross-sectional view of an implantable creatinine sensor in accordance with various embodiments of the invention.

FIG. 6 is a schematic cross-sectional view of an implantable creatinine sensor in accordance with various embodiments of the invention.

FIG. 7 is a schematic cross-sectional view of an implantable creatinine sensor in accordance with various embodiments of the invention.

FIG. 8 is a schematic view of an implantable creatinine sensor in accordance with various embodiments of the invention.

FIG. 9 is a block diagram of an implantable medical device (IMD) with a creatinine sensor in accordance with various embodiments of the invention.

FIG. 10 is a block diagram of an implantable medical device (IMD) with a creatinine sensor in accordance with various embodiments of the invention.

FIG. 11 is a perspective view of an implantable medical device (IMD) with an integrated chemical sensor in the header.

FIG. 12 is a perspective view of an implantable medical device (IMD) with an integrated chemical sensor disposed on the device housing.

FIG. 13 is a schematic view of a medical system in accordance with an embodiment of the invention.

FIG. 14 is a schematic view of a medical system in accordance with an embodiment of the invention.

FIG. 15 shows creatinine deiminase enzymatic assay results comparing the activity of bound and unbound creatinine deiminase.

FIG. 16 shows 8-hydroxypyrene-1,3,6-trisulfonic acid (HPTS) absorbance, before and after addition of a 5 mM creatinine solution with creatinine deiminase bound beads.

FIG. 17 shows HPTS absorbance in creatinine solutions of varying concentrations with unbound creatinine deiminase.

While the invention is susceptible to various modifications and alternative forms, specifics thereof have been shown by way of example and drawings, and will be described in detail. It should be understood, however, that the invention is not limited to the particular embodiments described. On the contrary, the intention is to cover modifications, equivalents, and alternatives falling within the spirit and scope of the invention.

### DETAILED DESCRIPTION OF THE INVENTION

Applicants have surprisingly discovered that creatinine, unlike glucose, passes across the fibrous capsule wall that forms around implanted devices in quantities sufficient so that concentrations of creatinine inside the capsule are essentially the same as concentrations within mixed venous blood. Specifically, Applicants have discovered that creatinine passes across the fibrous capsule wall in sufficient amounts to allow for the use of a chronically implantable sensor to measure in vivo creatinine concentrations without the issue of chronic signal drift that plagues implantable glucose sensors. As such, embodiments of the invention include implantable creatinine sensors that can measure serum creatinine levels. The fact that the creatinine sensor is implantable is advantageous in that it allows creatinine concentrations to be measured as frequently as desired by clinicians, without requiring the patient to visit a care facility for blood draws.

Embodiments of the invention can include creatinine sensors that utilize the enzyme creatinine deiminase in order to detect creatinine. The term “creatinine deiminase enzyme” refers to one or more enzymes from a family of enzymes that have the activity of catalyzing a chemical reaction as shown below:

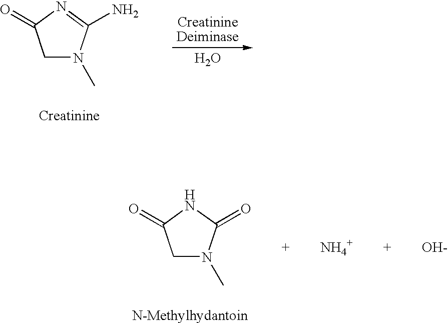

In this reaction, creatinine is turned into reaction products that have the effect of increasing the pH (making the pH more alkaline). The term “creatinine deiminase” is used herein interchangeably with the term “creatinine deaminase”. It will be appreciated that creatinine deiminase enzymes can be derived from various species including Bacillus sp. CNi-1365, Corynebacterium glutamicum, and Mesorhizobium loti, amongst others.

As shown above, creatinine deiminase can be used to convert creatinine into reaction products including N-methylhydantoin, ammonia, and hydroxide ion. These reaction products have the effect of increasing the alkalinity (increasing the pH) locally. Sensors of the invention can include a pH sensitive indicator compound. Local changes in pH can result in changes in the optical properties of the pH sensitive indicator, which in turn can be detected and processed in order to derive creatinine concentration.

While not intending to be bound by theory, creatinine sensors including creatinine deiminase offer various advantages. As one example, creatinine deiminase requires no co-factors for its catalytic activity, rendering the resulting sensor more robust.

In addition, embodiments of the invention including creatinine deiminase can sense creatinine without requiring the activity of other types of enzymes. That is, the reactions products of creatinine as catalyzed by creatinine deiminase can be sensed directly, without further enzymatically catalyzed reactions taking place. As such, the resulting sensor is more robust because the sensor is only dependent on the activity of one type of enzyme.

In some embodiments, the implantable creatinine sensor can include a creatinine deiminase enzyme that is immobilized. Immobilization can be accomplished using various techniques such as trapping or encapsulating the enzyme within a three-dimensional polymeric matrix. Immobilization can serve to prevent the enzyme from leaching out of the sensor over time. However, while not intending to be bound by theory, some forms of immobilization do not serve to increase the half life of the enzyme being used. For example, merely trapping or encapsulating an enzyme within a three-dimensional matrix would generally not be expected to prevent denaturation of an enzyme. In addition, trapping or encapsulation of an enzyme can sometime undesirably inhibit the diffusion of a target molecule, such as creatinine, into contact with the enzyme, reducing the sensitivity of the assay.

Covalent binding of an enzyme to a substrate is a particular form of immobilization that can be used to prevent enzyme molecules from leaching out of a sensor over time. As such, in some embodiments, implantable creatinine sensors of the invention can include a creatinine deiminase enzyme that is covalently bound to a substrate, such as a polymer matrix. Unlike some other forms of immobilization, covalent binding of the enzyme can also increase the half-life of the enzyme rendering it more practical for use with chronically implanted medical devices. For example, covalent binding of an enzyme can prevent denaturation of the enzyme thereby increasing its half-life.

Various approaches to the covalent binding of an enzyme exist and can be used depending on the chemistry of the particular type of enzyme. In the context of creatinine deiminase, the enzyme includes a significant number of lysine and arginine residues, each of which contain an amine functional group when the residues are part of a polypeptide. As such, in an embodiment, covalent bonds to the creatinine deiminase enzyme are formed through the amine groups on lysine and arginine residues. It will be appreciated that many different reactions can be used in order to form a covalent bond to a substrate through an amine functional group. As one example, an azlactone functional group can react with an amine functional group in order to form an amide linkage.

While not intending to be bound by theory, amide linkages can be desirable because of their relative stability. Stability is particularly important in the context of chronically implantable sensors. In addition, reactions between azlactone functional groups and amine groups to form amide linkages can be carried out under relatively mild conditions preserving activity of the enzyme. The following chemical reaction illustrated formation of an amide linkage between an azlactone group and an amine group.

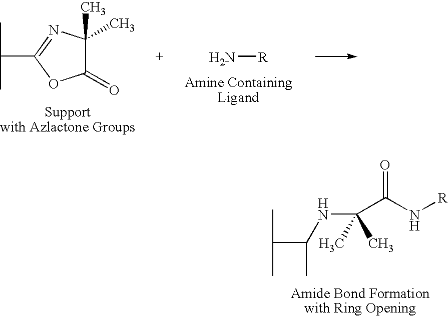

However, it will be appreciated that other types of bonds and bonding chemistries can also be used in order to bind an enzyme to a substrate.

Depending on the technique used for covalent bonding and the nature of the enzyme, covalent binding of an enzyme can reduce its activity to undesirable levels. However, as shown below in the examples, creatinine deiminase can be covalently bound to a substrate while preserving a significant amount of its native activity. Further, substantial activity was shown to be maintained over an extended period of time. Various details of exemplary creatinine sensors will now be described in greater detail.

Referring now to FIG. 1, a schematic cross-sectional view of an implantable creatinine sensor 100 is shown in accordance with an embodiment of the invention. The creatinine sensor 100 has a sensing element 110, including a recognition element 102 and transducing element 104. The recognition element 102 includes a creatinine deiminase enzyme. In some embodiments, the creatinine deiminase enzyme is covalently bound to a substrate. Exemplary substrate materials can include metals, polymers, ceramics, and the like. In some cases the substrate can be in the form of a three dimensional matrix. The creatinine deiminase enzyme can be covalently bound in a manner so as to preserve its activity. The recognition element 102 is configured to be in communication with bodily fluids in vivo, directly or indirectly. Specifically, the creatinine sensor 100 is configured so that creatinine in bodily fluids can contact the recognition element 102.

The transducing element 104 can be in ionic communication with the recognition element. The transducing element 104 can include a pH indicator, such as a pH sensitive compound. In some embodiments, the pH sensitive compound can be colorimetric or fluorimetric. In some embodiments, the pH sensitive compound can be immobilized. As such, an optical property of the transducing element 104 can change in response to a pH change that is caused by a reaction of creatinine in the recognition element.

It will be appreciated that various pH indicators can be used. By way of example, exemplary pH indictors can include congo red, neutral red, phenol red, methyl red, lacmoid, tetrabromophenolphthalein, α-napholphenol, 3-octadecanoylumbelliferone, 5(6)-carboxynaphthofluorescein, 7-hydroxycoumarin-3-carboxylic acid, 8-hydroxypyrene-1,3,6-trisulfonic acid trisodium salt, 8-hydroxypyrene-1,3,6-trisulfonic acid (HPTS), N-octadecanoyl-Nile blue, Rhodamine B octadecyl ester perchlorate, and the like. In some embodiments, the pH indicator has a pKa of between about 5 and 8. In some embodiments, the pH indicator can include a dye with internal referencing capability. By way of example the dye can be one where pH can be assessed as a ratio of absorbance or emission at one wavelength in comparison to absorbance or emission at another wavelength.

The pH indicator can optionally be appended with one or more organic substituents chosen to confer desired properties useful in formulating the transducing element. By way of example, the substituents can be selected to stabilize the pH indicator with respect to leaching into the solution to be sensed, for example, by incorporating a hydrophobic or polymeric tail or by providing a means for covalent attachment of the pH indicator to a polymer support within the transducing element.

### Non-Carrier Based Transducing Elements

In some embodiments, the transducing element 104 can be a non-carrier based transducing element. Non-carrier based transducing elements can include a hydrophilic pH indicator dye that is covalently attached to a hydrophilic polymer matrix (substrate), and which selectively responds to pH changes within the creatinine sensor to directly produce either a colorimetric or fluorescent response. In an embodiment of a non-carrier transducing element, a pH indicator is covalently bonded to a suitable substrate. As an example, hydroxypyrene trisulfonate can be covalently attached to an azlactone functional hydrophilic porous polyethylene membrane or to an azlactone functional beaded support to produce a fluorescence based optical pH sensor. As another example, hydroxypyrene trisulfonate, can be covalently attached to an amine functional cellulose membrane or bead to produce a fluorescence-based pH non-carrier ion sensor. The fluoroionophore can be covalently bonded to a substrate by any useful reactive technique, which may depend upon the chemical functionality of the particular pH indicator. The substrate can, in turn, be attached to a backing membrane or layer.

A specific example of a non-carrier based transducing element includes a sensing layer that includes hydroxypyrene trisulfonate covalently bonded to a crosslinked amine functional cellulose membrane (CUPROPHAN™; Enka AG, Ohderstrasse, Germany), the sensing layer being adhered to a polycarbonate backing membrane by FLEXOBOND 430™ urethane adhesive and the backing membrane having coated thereon CW14™ pressure-sensitive adhesive on a release liner. Another specific example of a non-carrier transducing element includes a sensing layer that includes hydroxypyrene trisulfonate covalently bonded to a crosslinked azlactone functional hydrogel with a linker such as a diamine linker. The sensing layer can then be photocrosslinked within the cavity of a substrate, such as a microwell, or the gel capsule of a satellite sensor. The term “satellite sensor” can be used to describe implanted chemical sensors that are remote from other implanted devices, such as remote from a pulse generator.

In various embodiments of non-carrier based transducing elements, the substrate can be a polymeric material that is water-swellable and permeable to the ionic species of interest, and insoluble in the medium to be monitored. Exemplary substrate materials include, for example, ion- and creatinine-permeable cellulose materials, high molecular weight or crosslinked polyvinyl alcohol (PVA), dextran, crosslinked dextran, polyurethanes, quaternized polystyrenes, sulfonated polystyrenes, polyacrylamides, polyhydroxyalkyl acrylates, polyvinvyl pyrrolidones, hydrophilic polyamides, polyesters, and mixtures thereof. In an embodiment, the substrate is cellulosic, especially ion- and creatinine-permeable crosslinked cellulose. In an embodiment, the substrate comprises a regenerated cellulose membrane (CUPROPHAN™, Eenka AG, Ohderstrasse, Germany) that is crosslinked with an epoxide, such as butanediol diglycidyl ether, further reacted with a diamine to provide amine functionality pendent from the cellulose polymer. In an alternate embodiment, the substrate comprises azlactone functional hydrophilic porous polypropylene that has been amine functionalized using a diamine functionality pendent to the azlactone.

Attachment of hydroxypyrene trisulfonate to an amine functional membrane or bead can be achieved using methods outlined in U.S. Pat. No. 5,591,400 (incorporated herein by reference) by converting hydroxypyrenetrisulfonate to acetoxypyrenetrisulfonate, reacting acetoxypyrenetrisulfonate with thionyl chloride and a catalytic amount of disubstituted formamide to form acetoxypyrenetris(sulfonyl)chloride, reacting acetoxypyrenetris(sulfonyl)chloride with the amine-modified polymeric ion-permeable matrix material to form bound acetoxypyrenesulfonamide; and converting the bound acetoxypyrenesulfonamide to the hydroxy form to form a pH responsive transducing element. Optionally, once the desired amount of the pH indicator is covalently bonded to the aminoethylcellulose, remaining amino groups are blocked by acylation.

In some embodiments, the non-carrier based transducing element can be prepared using commercially available polymer supported pH indicators such as dextran-hydroxypyrene conjugate or dextran-Texas Red conjugate, both commercially available from Molecular Probes-Invitrogen (Carlsbad, Calif.).

In some embodiments, the non-carrier based transducing element can be prepared utilizing a photocrosslinkable hydrogel having reactive functional groups for covalently attaching chemically functionalized pH indicators. In an embodiment, the photocrosslinkable hydrogel can include azlactone functional copolymers. The azlactone functional copolymers can be crosslinked (cured) using photocrosslinking agents such as bisazides, bisdiazocarbonyls, and bisdiazirines. This type of crosslinking does not affect the azlactone groups, but creates a three dimensional hydrogel matrix. The azlactone functional polymers can then be reacted with pH indicator having reactive functional groups (such as primary amines, secondary amines, hydroxyl groups, and thiol groups). The reactive functional groups then react, either in the presence or absence of suitable catalysts, with the azlactones by nucleophilic addition to produce a covalent bond. The covalent bonding step can be carried out before or after coating, before or after curing, and before or after patterning.

### Carrier Based Transducing Elements

In other embodiments, the transducing element 104 can be a carrier based transducing element. Carrier based transducing elements include a compound, referred to as a chromoionophore, that reversibly exchanges protons within a hydrophobic matrix. The chromoionophore is a lipophilic fluorescent or colorimetric indicator dye. The chromoionophore can be dispersed in, and/or covalently attached to, a hydrophobic organic polymeric matrix. In operation, protons are reversibly sequestered by the chromoionophore within the organic polymer matrix giving rise to a color or fluorescence change. To maintain charge neutrality within the polymer matrix, sodium ions are reversibly released from a saturated ionophore within the matrix.

A specific example of a carrier based pH sensor includes a lipophilic anion exchanger NaHFPB: sodium tetrakis[3,5-bis(1,1,1,3,3,3-hexafluoro-2methoxy-2-propyl)phenyl]borate, sodium ionophore bis[(12-crown-4)methyl]2-dodecyl-2-methylmalonate, and pH sensitive chromoionophore III: 9-(diethylamino)-5-(octadecanoylimino)-5H-benzo[a]phenoxazine dispersed in a polymer matrix made from polyvinylchloride and bis(2-ethylhexyl)sebacate surfactant to produce a colorimetric pH sensor as a transducing element.

The hydrophobic organic polymeric matrix can include materials with sufficient tensile strength, chemical inertness, and plasticizer compatibility. Exemplary materials can include poly(vinyl chloride), derivatives of polyvinyl chloride, polyurethane, silicone rubbers, polyalkylmethacrylates, and polystyrene.

In an embodiment, the hydrophobic organic polymer matrix is made permeable to the analyte of interest with plasticizers. Suitable plasticizers can include 2-nitrophenyl octyl ether (NPOE), dioctyl sebacate (DOS), bis(2-ethylhexyl)sebacate (BEHS), dibenzyl ether (DBE), and the like. However, it is known that plasticizers can leach out of the hydrophobic organic polymer matrix over time. This may lead to decreased functioning of the sensor. Accordingly, in some embodiments, the sensing element includes a polymeric matrix that is self-plasticizing. Such polymers can include polyurethanes, polysiloxanes, silicone rubber, polythiophenes, epoxyacrylates, and methacrylic and methacrylic-acrylic copolymers. In an embodiment, ion selective polymer materials are produced with an acrylate backbone and a plurality of pendant lipophilic plasticizing groups derived from acrylate co-monomers. The lipophilic plasticizing groups can, for example be a pendant C₃₋₇ alkyl group that renders the polymer matix inherently soft (e.g. a glass transition temperature (Tg) of less than −10° C.) and does not require additional plasticizers, i.e. the polymer is in effect self-plasticizing, so that the problem of leaching of the plasticizer does not arise.

Specific lipophilic anion exchangers useful in a carrier based pH sensor can include sodium tetrakis(4-chlorophenyl)borate), designated NaTpClPB; sodium tetrakis[3,5-bis(1,1,1,3,3,3-hexafluoro-2methoxy-2-propyl)phenyl]borate, designated NaHFPB; sodium tetrakis[3,5-bis(trifluoromethyl)phenyl]borate, designated NaTFPB; sodium tetrakis(4-fluorophenyl)borate, combinations thereof, and the like.

The chromoionophore in a carrier based pH sensor can include congo red, neutral red, phenol red, methyl red, lacmoid, tetrabromophenolphthalein, α-napholphenol, and the like. The chromoionophore can be immobilized by covalent bonding to the polymer matrix. The chromoionophore can be dissolved into the polymer matrix with the aid of a plasticizer as described above.

Exemplary pH responsive chromoionophores can include Chromoionophore I, (9-(diethylamino)-5-(octadecanoylimino)-5H-benzo[a]phenoxazine), “ETH 5294” CAS No. 125829-24-5; Chromoionophore II, (9-diethylamino-5-[4-(16-butyl-2,14-dioxo-3,15 ioxaeicosyl)phenylimino]benzo[a]phenoxazine), “ETH 2439” CAS No. 136499-31-5 ; Chromoionophore III, (9-(diethylamino)-5-[(2-octyldecyl)imino]benzo[a]phenoxazine, “ETH 5350” CAS No. 149683-18-1; Chromoionophore IV, (5-octadecanoyloxy-2-(4-nitrophenylazo)phenol), “ETH 2412” CAS No. 124522-01-6; Chromoionophore V, (9-(diethylamino)-5-(2-naphthoylimino)-5H-benzo[a]phenoxazine), CAS No. 132097-01-9; Chromoionophore VI, (4′,5′-dibromofluorescein octadecylester), “ETH 7075” CAS No. 138833-47-3; Chromoionophore XI, (fluorescein octadecyl ester), “ETH 7061” CAS No. 138833-46-2.

The implantable creatinine sensor 100 can also include an optical excitation assembly 106 and an optical detection assembly 108. The optical excitation assembly 106 can be configured to illuminate the transducing element 104. By way of example, the optical excitation assembly can include a light-emitting diode (LED). In some embodiments, the excitation assembly 106 includes solid state light sources such as GaAs, GaAlAs, GaAlAsP, GaAlP, GaAsP, GaP, GaN, InGaAlP, InGaN, ZnSe, or SiC light emitting diodes or laser diodes that excite the sensing element(s) at or near the wavelength of maximum absorption for a time sufficient to emit a return signal. In other embodiments, the excitation assembly 106 can include other light emitting components including incandescent components. In some embodiments, the excitation assembly 106 can include a waveguide. The excitation assembly 106 can also include one or more band pass filters and/or focusing optics.

In some embodiments, the excitation assembly 106 includes a plurality of LEDs with band pass filters, each of the LED-filter combinations emitting at a different center frequency. According to various embodiments, the LEDs operate at different center-frequencies, sequentially turning on and off during a measurement, illuminating the sensing element. As multiple different center-frequency measurements are made sequentially, a single unfiltered detector can be used.

The optical detection assembly 108 can be configured to receive light from the transducing element 104. By way of example, in some embodiments, the detection assembly 108 includes a charge-coupled device (CCD). In other embodiments, the detection assembly can include a photodiode, a junction field effect transistor (JFET) type optical sensor, or a complementary metal-oxide semiconductor (CMOS) type optical sensor. In an embodiment, the detection assembly 108 includes an array of optical sensing components. In some embodiments, the detection assembly 108 can include a waveguide. The detection assembly 108 can also include one or more band pass filters and/or focusing optics. In an embodiment, the detection assembly 108 includes one or more photodiode detectors, each with an optical band pass filter tuned to a specific wavelength range. The optical detection assembly 108 can then generate a signal regarding the bandwidth and intensity of light that it is receiving. This signal can then be processed in order to derive information regarding the concentration of creatinine in the bodily fluid.

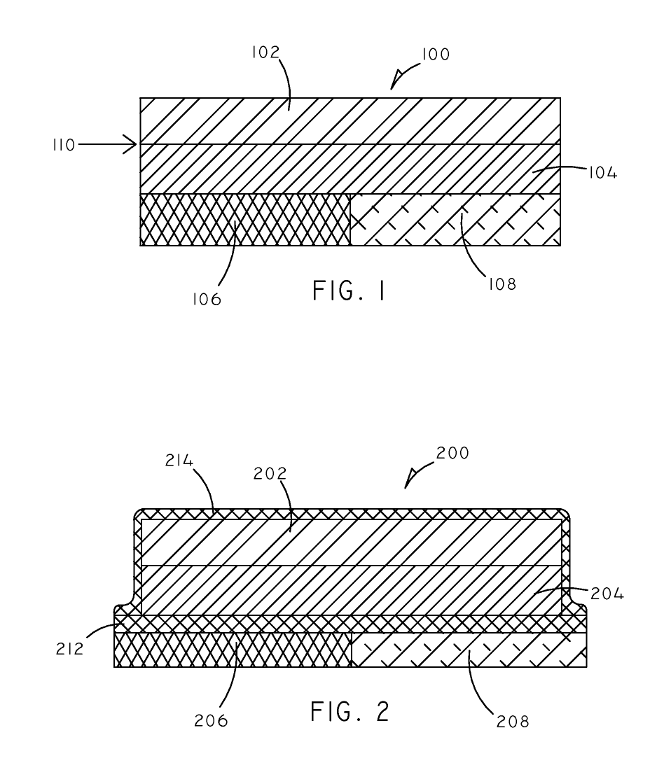

It will be appreciated that implantable creatinine sensors of embodiments herein can take on many different configurations. Referring now to FIG. 2, a schematic cross-sectional view of a creatinine sensor 200 is shown in accordance with another embodiment of the invention. The creatinine sensor includes a recognition element 202 and a transducing element 204, together forming a sensing element.

The sensor 200 further includes an overcoat layer 214, covering the sensing element. In some embodiments, the overcoat layer 214 can include hydrophilic polymers. Various types of polymers can be used. By way of example, the overcoat layer can include one or more of cellulose, polyvinyl alcohol, dextran, polyurethanes, quaternized polystyrenes, sulfonated polystyrenes, polyacrylamides, polyhydroxyalkyl acrylates, polyvinyl pyrrolidones, polyamides, polyesters, and mixtures and copolymers thereof.

Creatinine can diffuse through the overcoat layer 214 in order to reach the recognition element 202 of the sensing element. The overcoat layer 214 can include a material that is permeable to creatinine. In some embodiments, the overcoat layer 214 can be impermeable to proteases. Proteases in the in vivo environment could degrade the creatinine deaminase enzyme, reducing the useful life of the sensor. However, in embodiments where the overcoat layer 214 is impermeable to proteases, such as by having pores that are too small for the passage of proteases, protease mediated degradation of creatinine deaminase can be prevented. Similarly, in some embodiments, the overcoat layer 214 can be impermeable to creatinine deaminase, preventing the enzyme from leaching out of the sensor. This can also function to increase the useful life of the sensor.

The overcoat layer 214 can be opaque so as to optically isolate the sensing element from the tissues surrounding the sensor 200 in vivo. Alternatively, a separate opaque layer can be disposed over or under the overcoat layer 214. The overcoat layer 214 can include a polymeric material with an opacifying agent. Exemplary opacifying agents can include carbon black, or carbon-based opacifying agents, ferric oxide, metallic phthalocyanines, and the like. In a particular embodiment, the opacifying agent is carbon black. Opacifying agents can be substantially uniformly dispersed in the overcoat layer 214, or in a separate layer, in an amount effective to provide the desired degree of opacity to provide the desired optical isolation.

The sensor 200 can also include an opaque ink coating applied using a variety of techniques, such as an inkjet technique or an ink-screening technique. The sensor 200 can also include a black membrane. For example, it can include a black DURAPORE® membrane (available from Millipore as a white membrane which is then treated with black ink).

The sensor 200 further includes an optically transparent backing layer 212. The backing layer 212 can be configured to provide support (e.g. stiffness and handling capability) to the sensor 200. The backing layer 212 can be transparent and essentially impermeable to, or much less permeable than the overcoat layer 214 to the solution in which creatinine is present, such as blood, interstitial fluid, or a calibrating solution. Useful materials of construction for this backing layer 212 include polymeric materials such as polyesters, polycarbonates, polysulfones including but not limited to polyethersulfones and polyphenylsulfones, polyvinylidine fluoride, polymethylpentenes, and the like. The backing layer 212 can also include glasses, ceramics, and the like.

The backing layer 212 can be adhesively bonded or thermally fused to an optical excitation assembly 206 and an optical detection assembly 208. In embodiments where the backing layer 212 is adhesively bonded, the bonding adhesive can be essentially transparent to light used in excitation of the transducing element 204 and to light emitted or reflected there-from. An exemplary adhesive is FLEXOBOND 431™ urethane adhesive (Bacon Co., Irvine, Calif.).

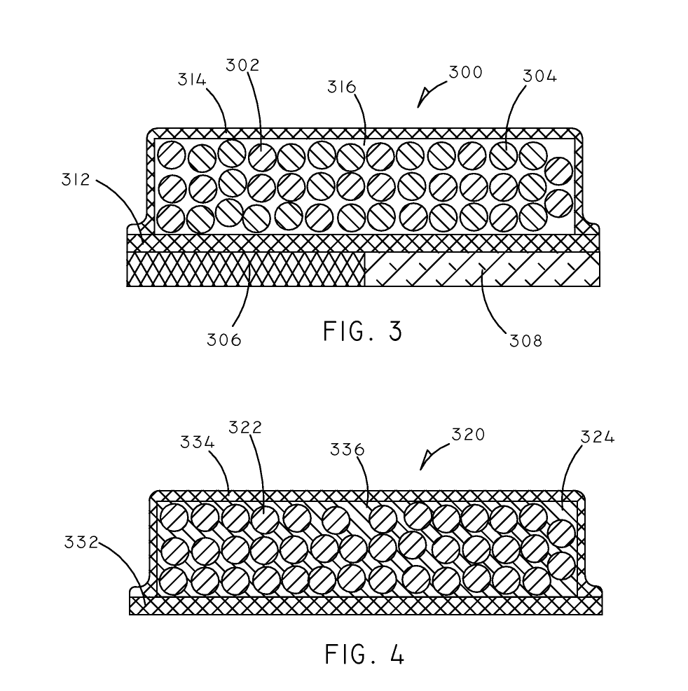

Referring now to FIG. 3, a schematic cross-sectional view of an implantable creatinine sensor 300 is shown in accordance with another embodiment of the invention. The creatinine sensor 300 includes an enclosed volume 316. In this embodiment, the sensing element is in the form of a plurality of beads 302 and 304. The beads include a first type of bead 302 that includes creatinine deiminase enzyme. The first type of bead 302 can be a recognition element. The beads also include a second type of bead 304 that includes a pH sensitive compound. The second type of bead 304 can be a transducing element. An overcoat layer 314 is disposed over the enclosed volume 316. The overcoat layer 314 can be optically opaque. In operation, creatinine can diffuse through the overcoat layer 314 and into the enclosed volume. The creatinine can interact with the creatinine deiminase enzyme and react to form reaction products that increase the pH inside the enclosed volume. The pH sensitive compound of the second type of bead 304 changes optical properties in response to the change in pH. For example, the pH sensitive compound exhibits a colorimetric or a fluorimetric response.

A backing layer 312 is disposed adjacent to the enclosed volume 316. An optical excitation assembly 306 is coupled to the backing layer 312 and configured to illuminate the beads within the enclosed volume 316. An optical detection assembly 308 is also coupled to the backing layer 312 and is configured to receive light from within the enclosed volume 316 that is either reflected or emitted.

Referring now to FIG. 4, a schematic cross-sectional view of an implantable creatinine sensor 320 is shown in accordance with another embodiment of the invention. The creatinine sensor 320 includes an enclosed volume 336. In this embodiment, the sensing element is in the form of a plurality of beads 322 and surrounded by a matrix 324. In some embodiments, the matrix 324 encapsulates the beads 322. The beads 322 include a creatinine deiminase enzyme. The beads 322 can be a recognition element. The matrix 324 includes a pH sensitive compound that changes optical properties in response to a change in pH. The matrix 324 can be a transducing element. An overcoat layer 334 is disposed over the enclosed volume 336. The overcoat layer 334 can be optically opaque. A backing layer 332 is disposed adjacent to the enclosed volume 336. In some embodiments, the overcoat layer 334 and the backing layer 332 are omitted.

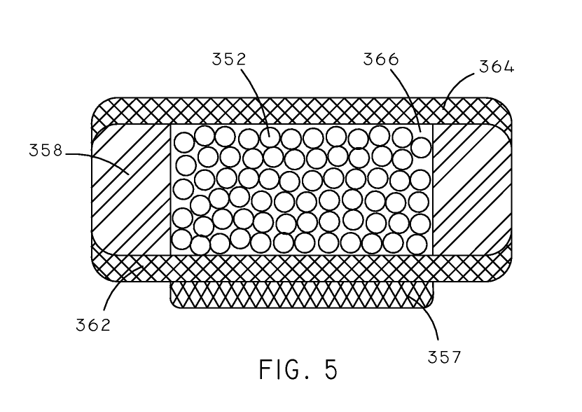

Referring now to FIG. 5, a schematic cross-sectional view of an implantable creatinine sensor is shown in accordance with another embodiment of the invention. The implantable sensor includes a frame element 358 defining an enclosed volume 366. The frame element 358 can provide a degree of structural support to the implantable creatinine sensor. A plurality of beads 352 are disposed within the enclosed volume 366. The beads 352 can include a creatinine deiminase enzyme and a pH sensitive compound. The enclosed volume is bounded by a top layer 364 and an optically transparent bottom layer 362. The top layer 364 can be optically opaque. An integrated optical interrogation module 357 can be disposed adjacent to the bottom layer 362. The integrated optical interrogation module 357 can include elements of both an optical excitation assembly and an optical detection assembly. Specifically, the optical interrogation module 357 can include elements that are configured to illuminate the enclosed volume and receive light from the enclosed volume.

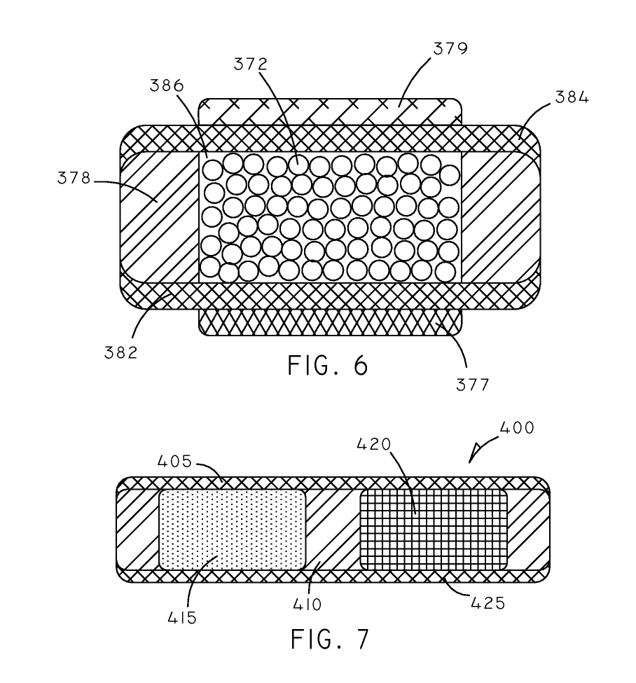

It will be appreciated that in some embodiments, an optical excitation assembly can be disposed on the opposite side of an implantable sensor from an optical detection assembly. Referring now to FIG. 6, a schematic cross-sectional view of an implantable creatinine sensor is shown in accordance with another embodiment of the invention. The implantable sensor includes a frame element 378 defining an enclosed volume 386. The frame element 378 can provide a degree of structural support to the implantable creatinine sensor. A plurality of beads 372 are disposed within the enclosed volume 386. The beads 372 can include a creatinine deiminase enzyme and a pH sensitive compound. The enclosed volume is bounded by a top layer 384 and an optically transparent bottom layer 382. The top layer 384 can be optically transparent. An optical excitation assembly 377 can be disposed adjacent to the bottom layer 382. The optical excitation assembly 377 can be configured to illuminate the enclosed volume 386. An optical detection assembly 379 can be disposed adjacent to the top layer 384. The optical detection assembly 379 can be configured to receive light from the sensing element.

FIG. 7 illustrates a cross-sectional view of a sensor 400 for measuring creatinine concentrations, according to various embodiments. The sensor 400 includes a first sensing element 415 and a second sensing element 420. In an embodiment, the first sensing element 415 can include a creatinine deiminase enzyme and a pH sensitive compound while the second sensing element 420 can be a creatinine insensitive element for optical referencing purposes, or more generally, a negative control.

The sensor 400 can include a cover layer 405. The cover layer 405 can include a creatinine permeable polymeric matrix. In some embodiments, the cover layer 405 can include hydrophilic polymers. Various types of polymers can be used. By way of example, the cover layer can include one or more of cellulose, polyvinyl alcohol, dextran, polyurethanes, quaternized polystyrenes, sulfonated polystyrenes, polyacrylamides, polyhydroxyalkyl acrylates, polyvinyl pyrrolidones, polyamides, polyesters, and mixtures and copolymers thereof.

The sensor can include a housing 410. The housing 410 can be configured to separate first sensing elements 415 and second sensing element 420. The housing 410 can include microwells or microcavities into which the first sensing element 415 and the second sensing element 420 fit. However, in some embodiments the sensing elements are disposed immediately adjacent to one another. The housing 410 can be constructed of various materials. In some embodiments, the housing 410 includes a polymeric matrix. In an embodiment, the housing 410 includes an ion permeable polymeric matrix.

The sensor can include a base layer 425 that is configured to be disposed between the sensing elements 415 and 420 and an optical detection assembly. In an embodiment, the base layer 425 is optically transparent over the wavelengths of interrogation and detection. Base layer 425 can be made of a variety of different materials including an optically transparent polymer, glass, crystal, etc. In some embodiments, base layer 425 is omitted such that components, such as the optical excitation assembly and/or optical detector assembly are in direct contact with indicator elements 415 and 420. The sensor can be configured to be mated to the header of a pulse generator, to an optical window in the housing of a pulse generator, to a lead, or to an optical window on a sensor in wireless communications with the pulse generator (satellite sensor).

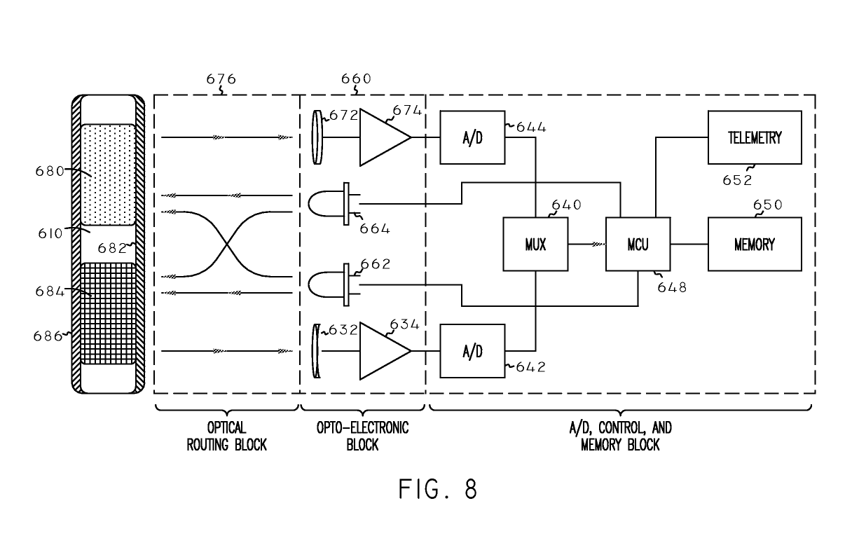

FIG. 8 is a schematic view of an implantable creatinine sensor, according to various embodiments. The sensor includes a first sensing element 680 and a second sensing element 684. The sensor includes a housing 610, a cover layer 686, and a base layer 682. In operation, the analyte of interest, for example creatinine, is present in a bodily fluid, for example interstitial fluid, in contact with the cover layer 686 of the sensor. The creatinine diffuses through the cover layer 686 and interacts with creatinine deiminase enzyme that is covalently bound within the first sensing element 680. The ensuing chemical reaction catalyzed by the enzyme increases the pH within the first sensing element. A pH sensitive compound in the first sensing element 680 exhibits an absorption spectral shift and/or a change in fluorescent intensity.

Sensing element 684 is also subject to the flux of creatinine diffusing through the cover layer 686. However, in this embodiment sensing element 684 is designed to be optically invariant to analyte concentrations and thus can serve as a negative control. In this embodiment, sensing element 684 can also be referred to as an optical reference element.

The diffuse reflectance spectra of sensing element 680 and optical reference element 684 are detected by first illuminating sensing element 680 and optical reference element 684 with emitters 662 and 664 (optionally in conjunction with optical filters.) Emitters 662 and 664, located within opto-electronic block 660, can be configured to produce light at two different center-wavelengths (e.g., wavelength 1 and wavelength 2 respectively) selected to effectively interrogate the spectral reflectance changes of sensing element 680 as the analyte concentration changes. Emitters 662 and 664 can be turned on and off alternately, under control of microprocessor control unit (MCU) 648 so that only one wavelength of reflectance is interrogated at a time. The light can be coupled from each of the emitters 662 and 664 to each of the sensing element 680 and the optical reference element 684 by means of an optical routing block 676. Diffusely reflected light, or emitted light in the case of a fluorescent sensor, is then routed from sensing element 680 and optical reference element 684, through optically transparent base layer 682, to sensor optical detector 672 and reference optical detector 632, respectively, by means of the optical routing block 676. The optical routing is achieved by means of optical fibers, waveguides, integrated optical packing of emitter and detector subassemblies, by free-space optics, or by other means known by those skilled in the art. The optical detectors 672 and 632 produce an electrical current that can be amplified by circuits 674 and 634 respectively to result in voltage signals indicative of the reflected light intensity returned from the sensing element 680 and the optical reference element 684 respectively. These analog voltage signals can then be processed by A/D converters 644 and 642, respectively, to produce digital signals and can then be routed through a multiplexer (MUX) 640. The resulting data is processed by MCU 648 and stored in memory 650 or routed to telemetry unit 652.

As the concentration of the analyte changes, the optical characteristics of sensing element 680 change, while the optical characteristics of the optical reference element 684 do not change. In one embodiment, MCU 648 can take the digitized emission or reflectance signal at a particular wavelength associated with excitation of the sensing element 680 and then calculate a corrected signal based upon the digitized signals associated with optical reference element 684. The MCU 648 can then take the digitized emission or reflectance signal at a second wavelength associated with excitation of the sensing element 680 and calculate a second corrected signal based upon the digitized signals associated with optical reference element 684. The MCU 648 can then use the ratio of corrected optical signals at the two wavelengths in estimating creatinine concentration. This ratio is processed by MCU 648 by a program routine or lookup table into representations of analyte concentration. Resulting data can be stored, transmitted to external devices, or integrated into the functions of an accompanying therapeutic device.

Embodiments of creatinine sensors of the invention may be calibrated initially and/or periodically after implantation to enhance accuracy. It will be appreciated that calibration can be performed in various ways. By way of example, after the creatinine sensor is implanted, blood can be drawn and creatinine concentration in the blood can be assessed using standard in vitro laboratory techniques. The concentrations indicated by the in vitro testing can then be compared with the concentrations indicated by the implanted device, and the implanted device can then be corrected (offset correction) based on the difference, if any. The offset correction value can be stored in circuitry and automatically applied to future measurements. In some embodiments, this correction procedure is performed after the foreign body response has formed a tissue pocket around the implanted device. In some embodiments, this correction procedure is performed at regular intervals.

The creatinine sensor can measure the concentration of creatinine at a programmable rate (using a programmable timer), according to an embodiment. The creatinine sensor can also be configured to measure concentrations of analytes on demand, as dictated by a program routine or as initiated by an externally communicated command. The creatinine concentration measurements can be equally spaced over time, or periodic, in an embodiment. For example, the optical excitation assembly can be configured to interrogate the sensing element periodically. The measurements can be taken at various times in a non-periodic manner, or intermittently, in an embodiment. In one embodiment, a measurement is made approximately once per hour.

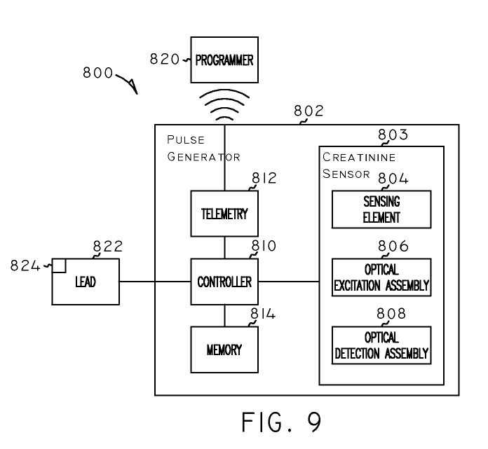

It will be appreciated that implantable creatinine sensors as described herein can be used in conjunction with various implantable medical devices. Referring now to FIG. 9, a block diagram of an implantable medical device (IMD) 802 with a creatinine sensor 803 is shown. In various embodiments, the IMD 802 can include a cardiac rhythm management device, such as a pacemaker, a cardiac resynchronization therapy (CRT) device, a remodeling control therapy (RCT) device, a cardioverter/defibrillator, or a pacemaker-cardioverter/defibrillator. One exemplary cardiac rhythm management device is disclosed in commonly assigned U.S. Pat. No. 6,928,325, issued Aug. 9, 2005, the contents of which is herein incorporated by reference. In the embodiment shown in FIG. 9, the creatinine sensor 803 is integrated with the IMD 802. The creatinine sensor 803 is configured to detect the concentration of creatinine in a bodily fluid. Bodily fluids can include blood, interstitial fluid, serum, lymph, and serous fluid. The creatinine sensor 803 includes a sensing element 804. The creatinine sensor 803 also includes an excitation assembly 806 and a detection assembly 808. The creatinine sensor 803 can be configured to operate in various ways including colorimetrically and/or fluorimetrically.

The excitation assembly 806 can be configured to illuminate the sensing element 804. The sensing element 804 can include a creatinine deiminase enzyme covalently bound to a substrate and a pH-indicating compound. Creatinine can diffuse into the sensing element 804 and result in a fluorimetric or colorimetric response. The detection assembly 808 can be configured to receive light from the sensing element 804. In an embodiment, the detection assembly 808 includes a component to receive light.

Embodiments of the invention can include an implantable medical device having a creatinine sensor co-located with a pulse generator body, located on a lead connected to a pulse generator body through a header, or separately located in a sensor module in wired or wireless communication with a pulse generator body.

The implantable medical device (IMD) 802 can include a pulse generator. The term “pulse generator” as used herein shall refer to the part or parts of an implanted system, such as a cardiac rhythm management system or a neurological therapy system, containing the power source and circuitry for delivering pacing and/or shock therapy. The pulse generator 802 can include a controller circuit 810 (including components such as a pulse generator circuit) to communicate with the creatinine sensor 803, a telemetry circuit 812 to communicate with the controller circuit 810 and an external module 820 (such as a programmer module or advanced patient management device), and a memory circuit 814 in communication with the controller circuit 810.

The implantable system 800 can include at least one implantable electrical stimulation lead 822 coupled to the pulse generator 802, the at least one implantable lead 822 configured to be connected to at least one implantable electrode 824 capable of electrically stimulating tissue. However, it will be appreciated that embodiments of the invention can also include implantable systems, such as cardiac rhythm management systems, that do not include electrical stimulation leads, such as leadless implantable cardioverter-defibrillators.

In various embodiments, the controller circuit, telemetry circuit, and memory circuit are within a device body or housing. In some embodiments, the creatinine sensor 803, or some of the components thereof, are disposed within the device body or housing. In some embodiments, the creatinine sensor 803, or some of the components thereof are disposed on the device body or in an aperture in the device body. In an alternative embodiment, the optical excitation assembly 806 and the optical detection assembly 808 can be disposed within the device body, while the sensing element 804 is disposed outside of the device body. In such an embodiment, optical communication between the optical excitation assembly 806, the sensing element 804, and the optical detection assembly 808 is maintained by waveguides, optical lenses, or optical windows. For example, an optical lens or an optical window (transparent member) can be disposed within an aperture on the device body, the sensing element can be optically coupled to the outside of the lens or window, and the optical excitation assembly and optical detection assembly can be optically coupled to the inside of the lens or window.

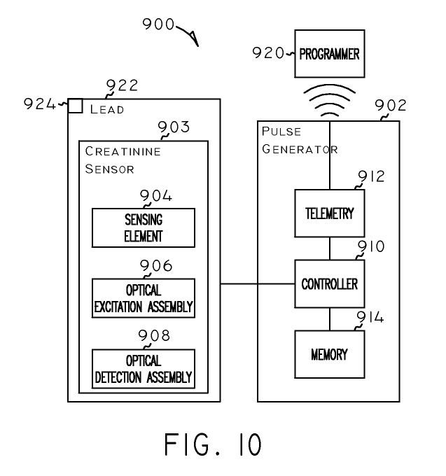

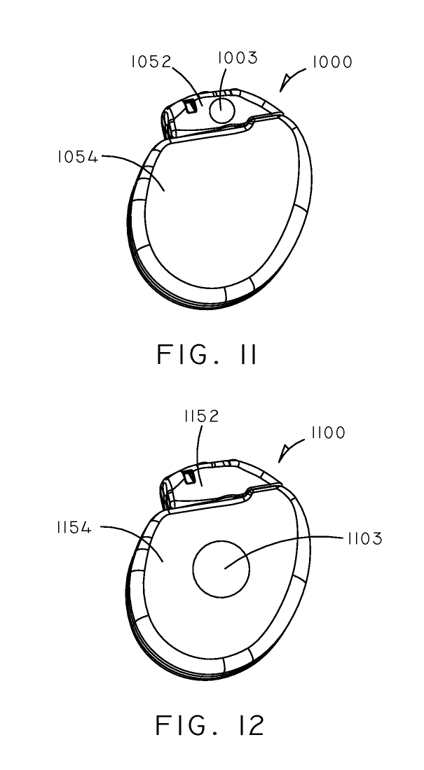

FIG. 10 illustrates an embodiment of an implantable system 900 having a pulse generator 902 coupled to (such as electrically or optically), but separate from, a creatinine sensor 903. The pulse generator 902 can include a controller circuit 910 to communicate with the creatinine sensor 903, a telemetry circuit 912 to communicate with the controller circuit 910 and an external module 920 (such as a programmer module), and a memory circuit 914 to communicate with the controller circuit 910. The creatinine sensor 903 includes a sensing element 904, an optical excitation assembly 906, and an optical detection assembly 908. The implantable system 900 can include at least one implantable lead 922 connected to the pulse generator 902, the at least one implantable lead 922 configured to be connected to at least one implantable electrode 924 capable of electrically stimulating tissue. The implantable system 900, can include a chemical sensing lead to electrically or optically couple the pulse generator 902 with the creatinine sensor 903.

FIG. 11 is a perspective view of an implantable medical device (IMD) 1000 with an integrated chemical sensor 1003 in the header 1052. The chemical sensor 1003 can be configured to detect creatinine. The IMD 1000 includes a housing or body 1054. In this embodiment, the chemical sensor 1003 is located in the IMD device header 1052 which is in turn coupled to the housing 1054.

FIG. 12 is a perspective view of an implantable medical device (IMD) 1100 with an integrated chemical sensor 1103 disposed on the device housing 1154. The chemical sensor 1103 can be configured to detect creatinine. The device housing 1154 is coupled to a device header 1152. In this embodiment, the chemical sensor 1103 is coupled to the device housing 1154. According to various embodiments, circuitry for correction of cardiac arrhythmias uses a common battery with the excitation assembly.

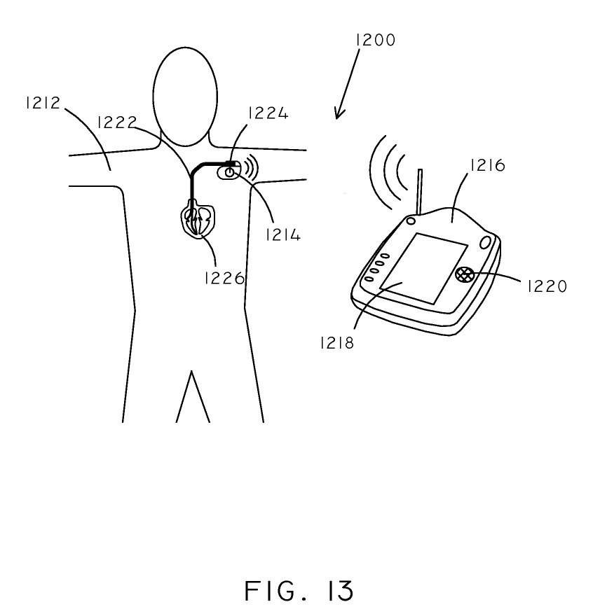

It will be appreciated that embodiments can include systems including an implantable creatinine sensor along with an external device, such as a patient management system. Referring now to FIG. 13, a schematic view of a system 1200 is shown including a creatinine sensor 1224 in accordance with an embodiment of the invention. The system 1200 includes an implantable device 1214 with an integrated creatinine sensor 1224 implanted within the body 1212 of a patient. In some embodiments, the implantable device 1214 can be an implantable cardiac rhythm management (CRM) device and can include electrical stimulation leads 1222 to deliver electrical stimulation pulses to the patient's heart 1226. By way of example, the device can be a pacemaker, a cardiac resynchronization therapy (CRT) device, a remodeling control therapy (RCT) device, a cardioverter/defibrillator, or a pacemaker-cardioverter/defibrillator.

The system 1200 can also include an external interface device 1216. The external interface device 1216 can include a video output 1218 and/or an audio output 1220. The external interface device 1216 can communicate with the implantable device 1214 wirelessly. The external interface device 1216 can take on many different forms. In some embodiments, the external interface device 1216 can include a programmer or programmer/recorder/monitor device. In some embodiments, the external interface device 1216 can include a patient management system. An exemplary patient management system is the LATITUDE® patient management system, commercially available from Boston Scientific Corporation, Natick, Mass. Aspects of an exemplary patient management system are described in U.S. Pat. No. 6,978,182, the contents of which are herein incorporated by reference. In some embodiments, the external interface device 1216 can include a hand-held monitoring device.

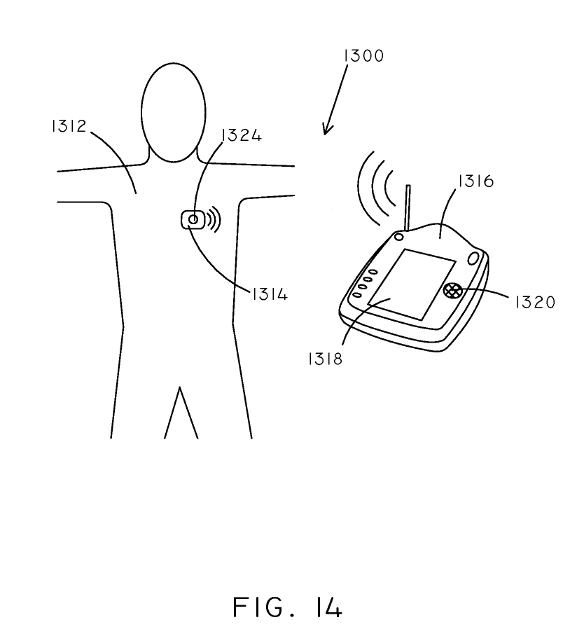

While the system of FIG. 13 depicts a creatinine sensor integrated with a medical device that can be, for example, a CRM device, it will be appreciated that embodiments of systems can also include a creatinine sensor that is not integrated with a CRM device. For example, the creatinine sensor can be integrated with a monitoring device or can be a stand-alone implanted sensor. Referring now to FIG. 14, a schematic view is shown of a system 1300 including a creatinine sensor 1324 in accordance with another embodiment of the invention. The system 1300 includes a monitoring device 1314 with a creatinine sensor 1324 implanted within the body 1312 of a patient. The system 1300 can also include an external interface device 1316. The external interface device 1316 can include a video output 1318 and/or an audio output 1320. The external interface device 1316 can communicate with the implantable device 1314 wirelessly. The external interface device 1316 can take on many different forms. In some embodiments, the external interface device 1316 can include a patient management system.

Aspects of the present invention may be better understood with reference to the following examples. These examples are not intended as limiting the scope of the invention.

### EXAMPLES

### Example 1

### Covalent Binding of Creatinine Deiminase

Azlactone functional support beads were purchased as UltraLink Biosupport from Pierce Biotechnology (Rockford, Ill.). 20 mg of azlactone beads were transferred to a 2-mL Handee Spin Cup Column with 1.5 mL of 2 mg/mL protein (bovine serum albumin (BSA) or creatinine deiminase (CD)) solution in 0.1 M MOPS, 0.6 M citrate coupling buffer (pH was adjusted to 7.5). The sample was briefly vortexed and then gently rocked for 2 hours at room temperature. The protein solution was then separated by draining it off the column (filtrate becomes unknown 1), and the beads were rinsed with PBS, which was also collected (filtrate becomes unknown 2). The mass of the filtrates were measured. A quench solution of 3.0 M ethanolamine (1.6 mL) was added to the beads to block any un-reacted azlactone sites. Again, the sample was briefly vortexed, and gently rocked for 2.5 hours at room temperature. The quench solution was separated, and then the beads were rinsed several times with PBS and 1.0 M NaCl. Rinsing the beads with a high concentrated salt solution precipitates any unbound protein adsorbed on the beads. The enzyme-functional beads were then re-suspended in PBS and stored at 4° C.

### Example 2

### Determination of Protein Binding

To determine the concentration of the protein in solution, the Bradford assay, which is a total protein assay, was performed. The procedure is based on the formation of a complex between the dye, Brilliant Blue G and proteins in solution. 0.1 mL of the protein solution sample (standard solutions for calibration or unknowns from the immobilization experiment) was added to 3 mL of Bradford Reagent in a polystyrene cuvette. The solution was gently shaken and left to incubate at room temperature for 10 minutes. Absorbance at a wavelength of 595 nm was measured using a spectrophotometer (Beckman Coulter DU530, Fullerton, Calif.). Protein solutions of known concentrations were used to obtain a calibration plot, which was then used to determine the concentration of the unknown sample.

Using the mass (or volume) of the filtrate, the amount of protein in solution, unbound protein, was determined, and by difference, the bound protein was then calculated. Knowing how much protein is bound, the beads were characterized by the coupling efficiency and protein loading. The coupling efficiency (in percent), is the ratio of bound protein to the total protein in solution. Protein loading (in mg of protein per mL of bead) is the ratio of bound protein to the total volume of bead. The maximum protein loading for BSA is 14.3 mg/mL based on the UltraLink® Biosupport technical specifications. The protein loading obtained from this immobilization experiment for BSA is between 9.0 and 15.1 mg/mL.

The coupling efficiency for BSA was between 32.6% and 37.3%, while that for creatinine deiminase was 45.2%. Table 1 below summarizes the results of the Bradford assay using only the first unknown filtrate from the immobilization assay.

TABLE 1

| | Creatinine | Bovine Serum Albumin | |
|---|---|---|---|
| Bradford Assay | Deiminase Sample 1 | Sample 1 | Sample 2 |
| Eluent Mass (g) | 1.109 | 1.898 | 1.770 |
| Beads Mass (mg) | 20.4 | 10.8 | 20.7 |
| Beads Volume (mL) | 0.163 | 0.086 | 0.166 |
| Absorbance | 0.906 | 1.702 | 1.698 |
| Concentration (mg/mL) | 1.215 | 1.421 | 1.416 |
| Unbound Protein (mg) | 1.347 | 2.697 | 2.507 |
| Bound Protein (mg) | 1.355 | 1.303 | 1.493 |
| Percent Coupling | 45.17% | 32.57% | 37.32% |

*Protein was prepared in 0.1 M MOPS, 0.6 M Citrate pH 7.5. Coupling was performed for 2 hrs. % Coupling is the ratio of the mass of bound protein to the mass of total protein. Loading is the ratio of bound proteins to the volume of the gel.

### Example 3

### Activity of Covalently Bound Creatinine Deiminase

A creatinine deiminase activity assay was performed to evaluate the activity of the bound enzyme. The enzymatic assay involves the reaction of creatinine deiminase with creatinine, breaking it down into N-methylhydantoin and ammonia. Then, a second reaction takes place converting ammonia and α-ketoglutarate into glutamate by glutamic dehydrogenase (GDH). In the process, a molecule of β-nicotinamide adenine dinucleotide phosphate (β-NADPH) is oxidized into β-NADP⁺.

Procedurally, a creatinine solution (2.4 mL, 50 mM in 50 mM phosphate buffer (PB), pH was adjusted to 7.5) was mixed with a β-NADPH (0.3 mL, 3 mM in PB), α-ketoglutarate (0.3 mL, 10 mM in PB), and GDH (0.05 mL, 1000 units/mL) in a cuvette. The solution was mixed by inversion, and let to equilibrate to room temperature (assay required equilibration to 37° C.). After about 7 min, creatinine deiminase (0.10 mL) was added to the creatinine solution. The solution was immediately mixed by inversion, and the A_{340 nm} was measured for 5 minutes. The rate of A_{340 nm} change, ΔA_{340 nm}/min, was determined using the maximum linear rate for both the test and blank, and then used in the calculation of the enzyme activity using the equation below:

$$units \text{/} mL enzyme = \frac{( Δ {A}_{340} / {min}_{Test} - Δ {A}_{340} / {min}_{Blank} ) ( 3.15 ) ( d f )}{( 6.22 ) ( 0.1 )}$$

where, ΔA₃₄₀/min is the maximum linear rate; 3.15 is the total volume in mL; df is the dilution factor; 6.22 is the millimolar extinction coefficient of β-NADPH at 340 nm; and 0.1 is the volume in mL of enzyme used.

After determining that there is CD on the beads, an enzymatic activity assay was performed. The activity of the enzyme is measured in units/mg protein, and a unit of CD will hydrolyze 1 μmol of creatinine to N-methylhydantoin and ammonia per minute at pH 7.5 at room temp in a coupled system with GDH. As shown below in Table 2 and Table 3, the activity values obtained from the assay were 2.3 to 6.1 units/mg protein for unbound creatinine deiminase, and 1.9 to 2.4 units/mg protein for bound creatinine deiminase.

TABLE 2
Enzyme Activities for Bound Creatinine Deiminase

| | | Conc. of | | | |
|---|---|---|---|---|---|
| Vol. Bead | Mass of | Enzyme | Vmax | Activity | |
| Sol. (uL) | Beads (mg) | (mg/mL) | (dAbs/dt) | units/mL | units/mg |
| 0 | 0.000 | 0.000 | 0.000 | 0.000 | 0.000 |
| 25 | 0.319 | 0.212 | −0.100 | 0.506 | 2.390 |
| 50 | 0.638 | 0.424 | −0.205 | 1.039 | 2.451 |
| 75 | 0.956 | 0.635 | −0.274 | 1.388 | 2.186 |
| 100 | 1.275 | 0.847 | −0.322 | 1.633 | 1.928 |

*Vmax is the maximum linear slope of the kinetic absorbance plot. Activity (units/mg) is normalized to the enzyme concentration of the sample.

TABLE 3
Enzyme Activities for Unbound Creatinine Deiminase

| | | |
|---|---|---|
| Conc. of | | |
| Enzyme | Vmax | Activity |

| (mg/mL) | (dAbs/dt) | units/mL | units/mg |
|---|---|---|---|
| 0.000 | 0.000 | 0.000 | 0.000 |
| 0.025 | −0.030 | 0.153 | 6.139 |
| 0.050 | −0.061 | 0.307 | 6.139 |
| 0.125 | −0.136 | 0.691 | 5.525 |
| 0.250 | −0.212 | 1.074 | 4.297 |
| 0.500 | −0.336 | 1.703 | 3.407 |
| 0.750 | −0.412 | 2.086 | 2.782 |
| 1.000 | −0.505 | 2.558 | 2.558 |
| 1.500 | −0.688 | 3.482 | 2.321 |

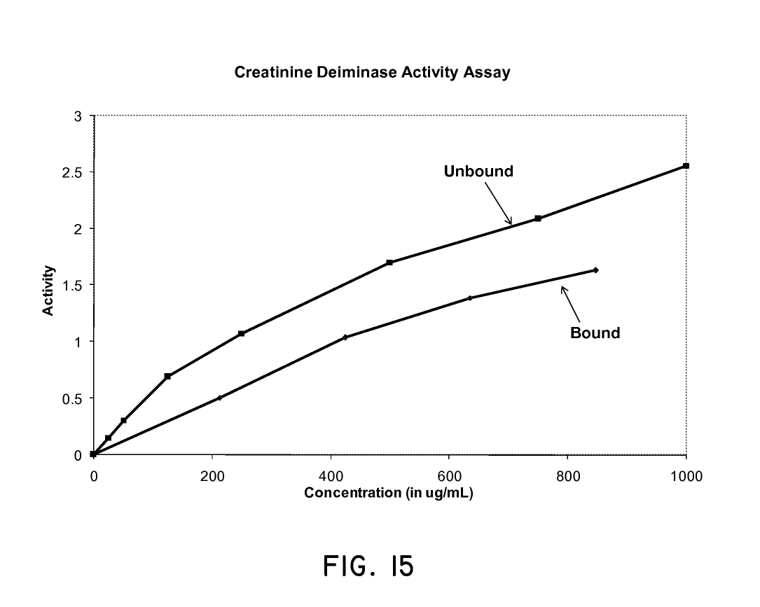

The activity of the bound creatinine deiminase is expected to be less than the unbound creatinine deiminase by about 50% due to diffusion limitations. The covalent attachment of creatinine deiminase to the azlactone beads limits the availability of binding sites for creatinine; in addition, by being on the support, it takes creatinine longer to bind to creatinine deiminase, which can affect the activity of creatinine deiminase. FIG. 15 is a plot of activity as a function of concentration of bound and unbound creatinine deiminase in solution. Based on the plot, the activity of the bound creatinine deiminase is about 70% of the unbound creatinine deiminase in solution. Also, it was determined that after a month in storage (in PBS and at 4° C.), the creatinine deiminase-bound beads were still active.

### Example 4

### Analysis of Test Solution Using Creatinine Deiminase Beads

To test the performance of the creatinine deiminase-bound beads, an assay was performed using 8-hydroxypyrene-1,3,6-trisulfonic acid (HPTS), which is an inexpensive, highly water-soluble pH indicator with a pKₐ of ˜7.3. HPTS exhibits a pH-dependent absorption shift that enables ratiometric measurements using an excitation ratio of 450/405 nm. Two different sets of experiments were performed. First, a solution of creatinine and HPTS (10 mM, 10 uL) in deionized water was prepared, and then creatinine deiminase (either on beads or in solution, 50 uL) was added. The other experiment involved a bolus of the creatinine solution. A solution of HPTS (10 mM, 10 uL) and creatinine deiminase (either on beads or in solution, 50 uL) in deionized water was prepared first, followed by the addition of creatinine. The absorbance between 300 and 650 nm were acquired before, immediately after, and 30 minutes after the addition of either creatinine deiminase or creatinine.

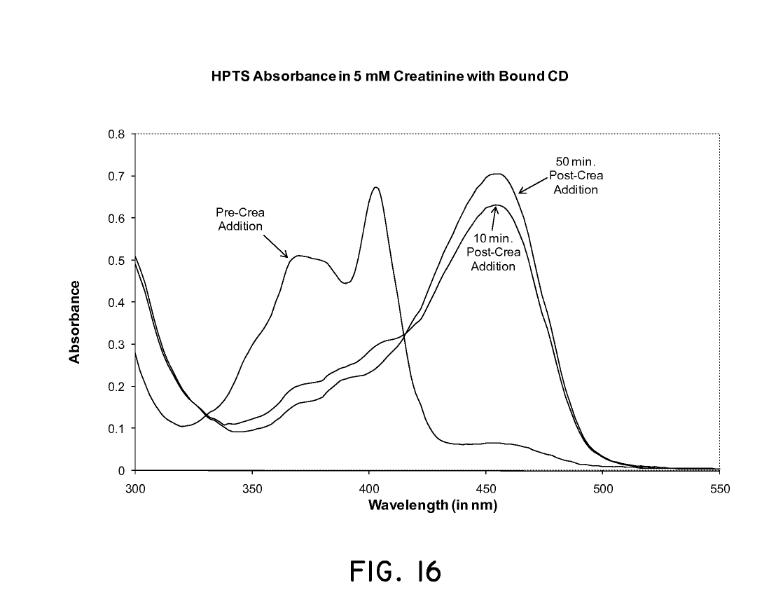

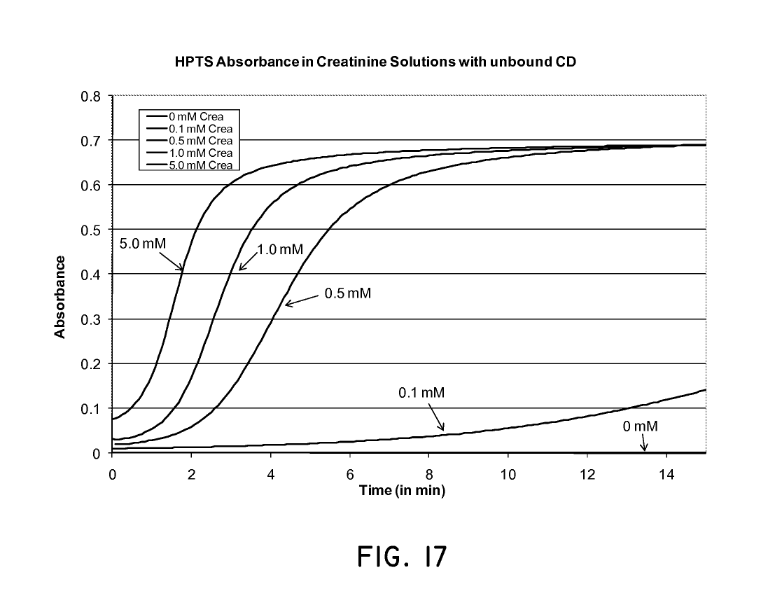

To evaluate the performance of the creatinine deiminase-bound beads, the beads were suspended in an HPTS solution, which shows if the active creatinine deiminase beads will actually bind creatinine and convert it to N-methylhydantoin and ammonia. The resulting ammonia should increase the pH, which consequently should cause a colorimetric change. FIG. 16 shows the effect of creatinine in solution with HPTS and creatinine deiminase -bound beads before, 10 min after, and 50 min after creatinine is added. The same behavior was observed in unbound creatinine deiminase. A short incubation time, which is dependent on the creatinine concentration, is required before the absorbance is measured. The maximum A_{450 nm} observed was around 0.7, which is when most of the HPTS molecules are converted to its basic form.

FIG. 17 illustrates the kinetic behavior of the reaction of creatinine with creatinine deiminase in HPTS solution. A_{450 nm} was measured for 15 min immediately after the addition of creatinine. With the same amount of creatinine deiminase in the system, the solutions with higher concentrations (>0.5 mM is elevated/pathologic serum creatinine level) reached the maximum A_{450 nm} faster than the solutions with lower concentrations (0.1 mM is normal serum creatinine level). Creatinine was broken down faster, and resulting ammonia elevated the pH to the maximum HPTS can detect. However, it may be different if this assay is to be performed in a system under flow, which is more representative of physiologic conditions.

### Example 5

### Creatinine Concentration Inside Fibrous Tissue Capsule

After implantation of a medical device, a fibrous tissue capsule forms around the implanted device as a result of wound healing phenomena and the host response to the implant. The objective of this study was to evaluate the potential effects of the fibrous tissue capsule surrounding an Implantable Cardiac Defibrillator (ICD) on creatinine concentrations immediately surrounding the device.

Ten canines were implanted with an ICD device and cared for in accordance with standard laboratory procedures. At the end of the study, fluid was collected from inside the implant encapsulation tissue (or pocket) with a needle mounted on a catheter. Specifically, concentrations of creatinine in this fluid was measured and compared with concentrations in corresponding blood serum. In three of the ten animals there was sufficient fluid (100 uL) to analyze. Creatinine concentrations were measured using an iSTAT blood analyte monitor (i-Stat Corporation, Princeton, N.J.). Serum samples were also drawn from the animals and similarly analyzed. The data is shown in Table 4 below. The data show that creatinine concentrations inside the capsule are roughly equivalent to creatinine concentrations in blood serum. This example shows that creatinine concentration can be accurately measured from within an encapsulation pocket.

TABLE 4

| | Pocket | | | Pre- or | Notes on |
|---|---|---|---|---|---|
| | Age | Creatinine (mg/mL) | | Post- | Pocket |
| ID | (Days) | Serum | Pocket Fluid | Euthanasia | Fluid |
| 1 | 74 | 0.8 | 0.6 | Post | Clear |
| | | | | | Amber |
| 2 | 53 | 1.2 | 1.2 | Post | Clear |
| | | | | | Amber |
| 3 | 58 | 1.3 | 1.3 | Post | Clear |
| | | | | | Amber |

### Example 6

### Kinetics of Creatinine Concentration Change Inside Fibrous Tissue Capsule

One canine was implanted with an ICD device and cared for in accordance with standard laboratory procedures. At various time points, fluid was collected from inside the implant encapsulation tissue (or pocket) with a needle mounted on a catheter and compared with corresponding blood serum. For one of these time points, the serum creatinine level was artificially elevated using an initial IV bolus followed by a constant rate infusion of a creatinine solution over a 90 minute period. Creatinine concentrations were measured using an iSTAT blood analyte monitor (i-Stat Corporation, Princeton, N.J.).

TABLE 5

| | | Time After | Creatinine | | | |
|---|---|---|---|---|---|---|
| | Pocket | Creatinine | (mg/mL) | | Pre- or | Notes on |
| | Age | Infusion | | Pocket | Post- | Pocket |
| ID | (Days) | (min) | Serum | Fluid | Euthanasia | Fluid |
| 4 | 106 | 0 | 0.8 | 0.6 | Post | Clear |
| | | | | | | Amber |
| 5 | 218 | 90 | 1.2 | 1.2 | Post | Clear |
| | | | | | | Amber |

The data show that creatinine concentrations inside the capsule are responsive to changes in creatinine concentrations in blood serum, even when the creatinine levels are significantly elevated relative to physiologically normal levels. This example further shows that creatinine concentration can be accurately measured from within an encapsulation pocket.

### Example 7

### Preparation of Creatinine Sensing Elements Using Plasticized PVC Polymer Beads in the Transducer Element

A: Preparation of Transducer Element Beads

Microscopic beads based on poly(vinyl chloride), PVC and bis(ethylhexyl)sebacate, BEHS are prepared using a spray dry method. A THF solution containing 1 wt. % PVC and 1 wt. % of BEHS is sprayed with a nebulizer under heated air stream from a heat gun and PVC/BEHS particles (2.5±1 um in diameter) are collected in a cyclone chamber.

50 mg of BEHS solution containing 0.5 mg of hydrogen ion selective chromoionophore III, 1.6 mg of NaHFPB and 22.3 mg of sodium ionophore, bis(12-crown-4) are added to 300 mg of the PVC/BEHS beads and thoroughly mixed, to form pH sensing microscopic beads. These beads are also sensitive to sodium ion, which is relatively constant in a physiological sample.

B: Suspension of Transducer Element and Recognition Element Beads in Hydrogel

To prevent conglomerating of the pH sensing beads, the beads are suspended and fixed in a hydrogel matrix. Two milligrams of the pH sensing beads are well mixed with 1 mg of PEG and 1 mg of aqueous monomer solution containing 30 wt. % of acrylamide, 1 wt. % of N,N′-methylene-bis-acrylamide and 0.5 wt. % of photoinitiator, Irgacure 2959. The suspension is placed in between two slide glasses and then photopolymerized upon UV light irradiation for 15 min.

This hydrogel is quite workable and is gathered into the microwell of a standard microwell plate reader. To this mixture is added 1 mg of hydrated creatinine deiminase functional recognition beads as formed in Example 1, with mixing.

C: Fabrication of a PolyHEMA-Based Sensor Body

HEMA (2-hydroxyethyl methacrylate) based sensor bodies are prepared by making a polymer plate using a photopolymerization method applied to the monomer solution in between two slide glasses separated by a spacer. To prevent strong adhesion of the resulting polymer to the glass surface, surface modified slide glasses with octadecylsilane are used. The slides (25×75×1 mm) are cleaned in a 1N HNO₃ solution at 70° C. for 2 hours and after cooling they are rinsed with Milli-Q water. After drying in an oven, cleaned slide glasses are placed into 1 L of toluene with 1.5 g of octadecyltrichlorosilane and heated under reflux for 6 hours. The thus surface modified slide glasses are washed by ethanol and Milli-Q water, and used as substrates for photopolymerization.

A solution of 80 wt. % HEMA, 8.0 wt. % PEGMA (poly(ethylene glycol) methacrylate), 2.0 wt. % DEGDMA (di(ethylene glycol)dimethacrylate), 9.8 wt. % deionized water and 0.2 wt. % Irgacure 651 are transferred into a mold consisting of two surface modified slide glasses separated by a spacer with 400 um thickness. The solution is polymerized to form a crosslinked hydrogel by exposure to low intensity 365 nm UV light (ca 2 mW/cm²) for 10 min. After polymerization, the thus prepared polyHEMA film is removed from the mold. To prepare wells in the polyHEMA film for each sensing capsule, an excimer laser is used with a mask made of a brass plate 200 um thick in which four holes 1 mm in diameter are linearly aligned with 1.3 mm distances in between holes to create sensor compartments in a single sensor body. After successful laser drilling, the polyHEMA film with wells is washed with deionized water.

D: Fabrication of Sensor Window Membrane

To prepare the sensor window membrane, a solution of 32.9 wt. % HEMA, 16.9 wt. % PEGMA, 50 wt. % deionized water and 0.2 wt. % Irgacure 651 is put into a mold consisting of two slide glasses having hydrophobic surfaces separated by a spacer with 16 um thickness. After polymerization by exposure to UV light for 12 minutes, one of the slide glasses in the mold is carefully removed. In this case, the polyHEMA window membrane with 16 um thickness remains on the surface of another slide glass.

E. Construction of Sensor Body with Wells

To adhere the sensor body to the window membrane, 10 uL of the above mentioned monomer solution is applied to the surface of the thus prepared window membrane on the slide glass and spread. The sensor body is then placed on the window membrane, covered with a slide glass and clamped with binder clips. By exposure to UV light for 15 min, the polyHEMA-based sensor body containing wells with sealed bottoms is successfully prepared.

F. Filling the Sensor Wells and Completing Construction of the Sensor

The thus prepared sensor body is placed on a slide glass with sealed bottoms down and fixed with Scotch tape at the edges of the sensor body. The hydrogel suspension of creatinine sensing beads mixed with pH sensing beads is stuffed into one of the three wells of the sensor body using a tiny glass rod under a stereo microscope. Into an adjacent well is stuffed a corresponding hydrogel suspension having only the pH sensing beads.

Another piece of window membrane on a slide glass with hydrophobic surface is then prepared by using the same method mentioned above. Ten uL of the monomer solution mentioned above is applied on the surface of the window membrane and then spread. After removing the tape, the sensor body stuffed with beads on the slide glass is covered with the thus prepared window membrane together with the slide glass and cramped with binder clips. By exposure to UV light for 15 min, all wells with beads in the sensor body are sealed with another window membrane. At this point the sensors are ready for testing.

G. Optical Response of Creatinine Sensor

Reflectance spectra of the optical creatinine sensor in PBS buffer at pH 7.4 are measured using a fiber optic spectrometer (e.g. BIF400 UV-VIS, Ocean Optics, CA). As creatinine concentration increases over the range of 0 mM to 10 mM Creatinine, reflectance at 505 nm (corresponding to the acidic form of chromoionophore III) decreases while the reflectance at 580 nm (corresponding to the basic form of chromoionophore III) increases. The pH sensing beads can be used as an optical reference for these measurements. Optionally, the ratio of the 505 and 580 nm reflectances can be used in calculating the creatinine concentration.

Alternatively, optical characterization of the films can be done via fluorescence spectroscopy. When the sensor is exposed to creatinine, the pH change in the creatinine sensing well leads to a measurable change in its fluorescence properties. Emission peaks are observed at 647 nm and 683 nm. The former corresponds to the protonated form of chromoionophore III, while the latter corresponds to the deprotonated form. When the concentration of creatinine in the sample increases, the protonated peak at 647 nm decreases and the deprotonated peak at 683 nm increases. It has been reported that ratiometric analysis can minimize the effects of photobleaching and variations in lamp intensity. Therefore the intensity ratio of the two peaks (647 and 683 nm) is used instead of the absolute fluorescence.

### Example 8

### Preparation of Creatinine Sensing Elements Using Dextran-HPTS Conjugate as the Transducer Element

A: Construction of the Creatinine Sensor

A solution of 0.1 mM Dextran-HPTS conjugate (10,000 MW from Molecular Probes) in PBS buffer pH 7.4 was prepared. 1 part (w/w) dextran-HPTS solution is mixed with 1 part hydrated creatinine-deiminase functional beads formed as described in Example 1. A pasteur pipette is used to transfer an amount of this solution sufficient to fill the microwell of a sensor body prepared as described in Example 7. A window membrane is affixed as described in Example 7.

B. Optical Response of Creatinine Sensor

Reflectance spectra of the optical creatinine sensor in PBS buffer at pH 7.4 is measured using a fiber optic spectrometer (e.g. BIF400 UV-VIS, Ocean Optics, CA). As creatinine concentration increases over the range of 0 mM to 10 mM Creatinine, reflectance at 405 nm (corresponding to the acidic form of HPTS) decreases while the reflectance at 460 nm (corresponding to the basic form of HPTS) increases. The ratio of the 405 and 460 nm reflectances can be used in calculating the creatinine concentration.

Alternatively, optical characterization of the sensor can be done via fluorescence spectroscopy. When the sensor is exposed to creatinine, the pH change in the creatinine sensing well leads to a measurable change in it fluorescence properties. Emission peaks are observed at 460 nm and 510 nm. The former corresponds to the protonated form of HPTS, while the latter corresponds to the deprotonated form. When the concentration of creatinine in the sample increases, the protonated peak at 460 nm decreases and the deprotonated peak at 510 nm increases. It has been reported that ratiometric analysis can minimize the effects of photobleaching and variations in lamp intensity. Therefore the intensity ratio of the two peaks (460 and 510 nm) is used instead of the absolute fluorescence.

### Example 9

### Preparation of Creatinine Sensing Elements Using an HPTS Conjugated Cellulose Membrane as the Transducer Element

A. Preparation of Planar Transducer Element

CUPROPHAN® cellulose sheets infiltrated with glycerol (Akzo Nobel Chemicals; Chicago, Ill.) are washed with deionized water (10 minutes) to remove the glycerol. Each sheet is stretched on a glass plate and dried at room temperature.

B. Crosslinking

A solution of 3 g of 50% NaOH solution and 85 g DMSO in 350 mL deionized water is prepared. 450 g of a 50% aqueous EGDGE solution is added and mixed. This crosslinking solution is poured onto the CUPROPHAN® sheets and retained for 1 hour followed by rinsing with deionized water.

C. HDA (1,6-Hexandediamine) Reaction

Crosslinked CUPROPHAN® membranes are immersed in a solution of 120 g 70% HDA in 2.0 L deionized water for 2 hrs, rinsed with deionized water to wash off excess HDA.

D. HPTS Coupling Reaction

Acetoxypyrenetri(sulfonyl)chloride (APTSC) is prepared according to the procedure described in U.S. Pat. No. 5,591,400. A dye solution is then prepared by dissolving 30 mg APTSC in 50 mL acetone. To this is added 25 mL of a mixture made from 3 parts 10 mM sodium carbonate and 1 part 10 mM sodium bicarbonate. To reduce the number of sulfonyl chloride reactive sites on the dye from three sites to near one site on average, this dye solution is aged for 10-12 minutes before reaction with the membrane. HDA-functionalized CUPROPHAN® sheets are removed from the deionized water, towel dried, cut into 5 cm×5 cm squares and immersed in the aged dye bath for various amounts of time depending on the intensity of the fluorescence wanted in the finished membrane. The membranes are then removed from the dye solution, and placed in a bath of 2.5% w/w sodium carbonate and 10% w/w sodium chloride in water (buffer A), which is held at 70° C. for 20 minutes. This step removes ionically bound dye from the membranes. The membranes are then removed from buffer A, rinsed with deionized water, blotted and then soaked in a solution of 20% v/v glycerol in 2.5% w/w sodium carbonate aqueous solution for 15 minutes. Optionally, to acetylate the unreacted amine groups on the surface of the membrane, the membranes are then allowed to react for 5 minutes with a solution of 120 mL acetic anhydride, 75 ml of triethylamine, 1.5 g of 4-dimethylaminopyridine, and 480 ml of tetrahydrofuran. The membranes are then removed and soaked in buffer A at 70° C. for 30 minutes. The membranes are then rinsed in deionized water and soaked in a solution of 20% glycerol in water and dried.

E. Construction of Creatinine Sensor

A 1 mm diameter fragment of the HPTS functional membrane is placed in the bottom of a well of a sensor body prepared according to Example 7. To this well was added a hydrogel suspension of creatinine sensing beads prepared according to Example 7. A window membrane is then applied as described in Example 7. At this point the sensor is ready for testing

F: Optical Response of Creatinine Sensor

GaN LEDs from Nichia Chemical Industries, Tokushima, Japan, or Toyoda Gosei Co., Ltd (under the brand name LEDTRONICS™) are disposed within an implanted device and configured to be amplitude modulated at a 30 kHz carrier frequency, with a burst duration of 0.2 seconds, a repetition rate of 5 seconds, and an average output power of 2.5 mW. The light is focused, passed through a bandpass excitation filter (e.g. 475 nm center frequency and transmits at 50% of peak transmission at wavelengths of 460 nm and 490 nm; available from SpectroFilm; Woburn, Mass.), and transmitted to the sensing element through the optical window in the pulse generator. The modulated fluorescent return is similarly collected and passed through a bandpass emission filter (e.g. 550 nm center frequency and transmits 50% of peak transmission at wavelengths of 515 nm and 585 nm such as is available from SpectroFilm). The filtered optical signal is then be focused onto the active region of an S1337-33-BR™ photodiode detector (available from Hamamatsu Corp.; Bridgewater, N.J.) housed within the pulse generator. A small fraction of the excitation light is directly routed to the detector assembly and attenuated with a neutral density filter to provide a reference optical signal from the LED. In addition, an electronic switch is used to alternately sample the detector photo current and a 30 kHz electrical reference signal from the frequency generator. The detector output is directed to an electronic circuit within the pulse generator or satellite sensor that converts the photocurrent from the photodiode detector to a voltage. A transimpedance preamplification stage converts a photocurrent or the reference electrical signal to a voltage using an operational amplifier circuit. The following stage is a two-stage Delyiannis-Friend style bandpass filter designed to band limit the noise power while further amplifying the signal. The amplified photosignal or reference electrical signal is then digitally sampled at 100 kHz and processed to obtain a fluorescence intensity that is indicative of analyte concentration. Optionally, a pH sensor signal is also sampled and used to correct for minor pH dependent variations in the creatinine sensor signal.

It should be noted that, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as “arranged”, “arranged and configured”, “constructed and arranged”, “constructed”, “manufactured and arranged”, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

This application is intended to cover adaptations or variations of the present subject matter. It is to be understood that the above description is intended to be illustrative, and not restrictive. The scope of the present subject matter should be determined with reference to the appended claims, along with the full scope of equivalents to which such claims are entitled.

What is claimed is:

1. A chronically implantable creatinine sensor comprising:
a sensing element comprising a creatinine deiminase enzyme covalently bound to a first substrate, and a pH-indicating compound in ionic communication with the creatinine deiminase enzyme, the sensing element configured to change optical properties in response to changes in creatinine concentrations in vivo;
an optical excitation assembly configured to illuminate the sensing element; and
an optical detection assembly configured to receive light from the sensing element.

2. The implantable creatinine sensor of claim 1, the pH sensitive compound comprising a colorimetric or fluorimetric pH sensitive compound.

3. The implantable creatinine sensor of claim 1, the pH sensitive compound immobilized within the sensing element.

4. The implantable creatinine sensor of claim 1, the pH sensitive compound comprising 8-hydroxypyrene-1,3,6-trisulfonic acid or a conjugate salt thereof.

5. The implantable creatinine sensor of claim 1, the creatinine deiminase enzyme covalently bound to the first substrate through one or more residues selected from the group consisting of lysine and arginine.

6. The implantable creatinine sensor of claim 1, the first substrate comprising a porous bisacrylamide-azlactone copolymer.

7. The implantable creatinine sensor of claim 1, further comprising a housing defining an enclosed volume, the first substrate comprising polymeric beads disposed within the enclosed volume.

8. The implantable creatinine sensor of claim 7, the housing having a first side and a second side, the second side opposite the first side, the optical excitation assembly and the optical detection assembly both disposed on the first side of the housing.

9. The implantable creatinine sensor of claim 7, comprising an opaque cover layer disposed on the second side of the housing, the opaque cover layer comprising a creatinine permeable polymeric matrix.

10. The implantable creatinine sensor of claim 1, comprising a cover layer disposed over the sensing element, the cover layer impermeable to proteins.

11. The implantable creatinine sensor of claim 1, the covalently bound creatinine deiminase enzyme comprising at least 50% of the activity of otherwise identical unbound creatinine deiminase enzyme.

12. The implantable creatinine sensor of claim 1, the pH-indicating compound bound to a second substrate.

13. The implantable creatinine sensor of claim 12, the second substrate comprising a hydrophilic polymeric matrix encapsulating the first substrate, the hydrophilic polymeric matrix permeable to creatinine.

14. The implantable creatinine sensor of claim 13, the second substrate comprising a hydrogel matrix and the first substrate comprising polymeric beads.

15. An implantable medical device comprising:
a pulse generator; and
a chemical sensor in communication with the pulse generator, the chemical sensor configured to detect creatinine concentration in a bodily fluid, the chemical sensor comprising:
a sensing element comprising creatinine deiminase covalently bound to a substrate, and a pH-indicating compound in ionic communication with the creatinine deiminase.

16. The implantable medical device of claim 15, the chemical sensor comprising a communication interface configured to communicate wirelessly with the pulse generator via a radio frequency link, an ultrasonic link, or an acoustic link.

17. The implantable medical device of claim 15, the chemical sensor comprising a light source, the light source comprising a light emitting diode.

18. The implantable medical device of claim 17, the chemical sensor comprising a light source, the light source comprising
a first light emitting diode; and
a second light emitting diode; the first and second light emitting diodes configured to emit light at different wavelengths.

19. The implantable medical device of claim 15, the chemical sensor comprising a detection assembly comprising a component selected from the group consisting of a photodiode, a charge-coupled device (CCD), a junction field effect transistor (JFET) optical sensor, and a complementary metal-oxide semiconductor (CMOS) optical sensor.

20. The implantable medical device of claim 15, the pulse generator comprising:
a pulse generation circuit; and
an implantable housing configured to encapsulate the pulse generation circuit, the chemical sensor coupled to the implantable housing.

21. The implantable medical device of claim 20, further comprising:
a cardiac pacing lead; and
a device header coupled to the implantable housing, the device header configured to provide an electrical connection between the cardiac pacing lead and the pulse generator;
the chemical sensor coupled to the device header.

22. The implantable medical device of claim 21, further comprising:
a cardiac pacing lead, the chemical sensor coupled to the cardiac pacing lead.

23. The implantable medical device of claim 21, the implantable medical device comprising a pacemaker, a cardiac resynchronization therapy (CRT) device, a remodeling control therapy (RCT) device, a cardioverter/defibrillator, a pacemaker-cardioverter/defibrillator, or a hemodynamic monitor.

24. A medical system comprising:
an external monitoring device; and
a chemical sensor in communication with the external monitoring device, the chemical sensor configured to detect creatinine concentration in a bodily fluid, the chemical sensor comprising:
a sensing element comprising creatinine deiminase covalently bound to a substrate, and a pH-indicating compound in ionic communication with the creatinine deiminase.
